(19) United States
(12) Patent Application Publication
(10) Pub. No.: US 2017/0254795 A1
(43) Pub. Date: Sep. 7, 2017

(54) CARDIAC PLATFORM FOR ELECTRICAL RECORDING OF ELECTROPHYSIOLOGY AND CONTRACTILITY OF CARDIAC TISSUES

(71) Applicant: Lawrence Livermore National Security, LLC, Livermore, CA (US)

(72) Inventors: Fang Qian, Santa Cruz, CA (US); Heather Ann Enright, Livermore, CA (US); Kristen S. Kulp, Livermore, CA (US); Erik V. Mukerjee, Dublin, CA (US); David Soscia, Livermore, CA (US); Elizabeth K. Wheeler, Livermore, CA (US)

(21) Appl. No.: 15/060,252

(22) Filed: Mar. 3, 2016

(51) Int. Cl. G01N 33/483; G01N 27/00; C12N 5/077

(52) U.S. Cl. CPC G01N 33/4833; C12N 5/0657; G01N 27/002; C12N 2506/03

(57) ABSTRACT

Disclosed here is a cardiac platform, comprising a substrate layer comprising a substrate and a plurality of micro-strain gauges and a plurality of microelectrodes disposed on the substrate, a patterned layer disposed on the substrate layer which insulates the micro-strain gauges and exposes the microelectrodes, and a plurality of pillars disposed on the patterned layer. Also disclosed is a method for detecting electrophysiology and contractility of cardiac cells or tissues, comprising providing a cardiac platform that further comprises cardiac cells or tissues disposed on the pillars, and detecting electrophysiology of the cardiac cells or tissues using the microelectrodes and detecting contraction force of the cardiac cells or tissues using the micro-strain gauges.

### BACKGROUND

Contraction, or beat, is the most basic function of heart. Heart contraction is triggered by action potentials which are correlated with many complex factors and organ systems in the body. The ability to measure the mechanical properties/activities along with electrophysiology of healthy and impaired cardiac cells/tissues can provide important insights in elucidating the fundamental biology in cardiac science, developing precise tissue models and investigating drug effects.

Currently, the most prevailing method of electrophysiology recording is through the use of planar microelectrode arrays (MEA), while the detection of cell contractions is through optical video recording followed by computer-based analysis. See Chen et al., J. Appl. Physiol., 104:218-223 (2008); Rodriguez et al., J. Biomechanical Eng., 136:051005 (2014); and Hayakawa et al., J. Mol. Cellular Cardiology, 77:178-191 (2014). The simultaneous recording of both electrophysiology and contraction using electrical devices has not yet been reported.

Moreover, the current methodology is more suitable for 2D-cultured cardiac cells/tissues, which usually form a monolayer on a 2D surface. The monolayer formed by 2D-cultured cardiac cells/tissues is often thin enough for light transmission. But 2D-cultured cells behave very differently from in vivo cells. See Baker et al., J. Cell Science, 125:1-10 (2012). 3D-cultured cells, on the other hand, resemble in vivo cells but are usually opaque to light. Therefore, a need exists for electrical detection of contraction force of 2D-cultured and 3D-cultured cardiac cells/tissues down to single-/sub-cellular resolution.

### SUMMARY

Disclosed here is a novel platform adapted for electrical recording of both electrophysiology and contraction of in vitro-cultured cardiomyocytes and cardiac tissues. Such platform can serve as a versatile toolset to study cardiomyocytes in both thin and thick tissues.

Therefore, one aspect of some embodiments of the invention described herein relates to a cardiac platform, comprising a substrate layer which comprises a substrate and a plurality of micro-strain gauges and a plurality of microelectrodes disposed on the substrate, a patterned layer disposed on the substrate layer which insulates the micro-strain gauges and exposes the microelectrodes, and a plurality of pillars disposed on the patterned layer.

In some embodiments, the substrate layer comprises microelectrodes adapted to detect cardiac electrophysiology. In some embodiments, the substrate layer comprises microelectrodes adapted to detect extracellular electric potential correlated with action potential generation.

In some embodiments, the substrate layer comprises micro-strain gauges adapted to detect contraction force transmitted through the pillars and the patterned layer.

In some embodiments, the cardiac platform comprises serpentine-shaped micro-strain gauges. In some embodiments, the cardiac platform comprises spiral-shaped or square-spiral-shaped micro-strain gauges. In some embodiments, the cardiac platform comprises zigzag-shaped micro-strain gauges. In some embodiments, the cardiac platform comprises sea-urchin-shaped micro-strain gauges. In some embodiments, the cardiac platform comprises serially-connected micro-strain gauges. In some embodiments, the cardiac platform comprises rosette-shaped micro-strain gauges. Various shapes/geometries of the micro-strain gauges are shown in FIGS. 3-4.

In some embodiments, the micro-strain gauges have an average or mean linewidth of about 1-100 μm, or about 2-50 μm, or about 2-10 μm, or about 10-25 μm, or about 25-50 μm (see FIGS. 5-7).

In some embodiments, the micro-strain gauges have an average or mean side length of about 20-5000 μm, or about 50-2000 μm, or about 100-1000 μm, or about 100-200 μm, or about 200-500 μm, or about 500-1000 μm (see FIGS. 5-7).

In some embodiments, the micro-strain gauges comprise at least one metal or metal compound. In some embodiments, the micro-strain gauges comprises at least two metals or metal compounds. In some embodiments, the micro-strain gauges comprise one or more transition metals. In some embodiments, the micro-strain gauges comprise one or more post-transition metals. In some embodiments, the micro-strain gauges comprise one or more of Ti, Au, Cr, Pt, Pd, Ni, and Al. In some embodiments, the micro-strain gauges comprise Ti. In some embodiments, the micro-strain gauges comprise Au. In some embodiments, the micro-strain gauges comprise Cr. In some embodiments, the micro-strain gauges comprise at least two of Ti, Au, and Cr (See FIGS. 8-9).

In some embodiments, besides the micro-strain gauges and the microelectrodes, the substrate layer further comprises additional sensors.

In some embodiments, the substrate is a coated substrate. In some embodiments, the substrate is a coated glass substrate. In some embodiments, the substrate is a coated Si substrate. In some embodiments, the substrate is a SiO2-coated Si substrate. In some embodiments, the substrate is a SiN-coated Si substrate.

In some embodiments, the substrate comprises a polymeric material coated on a planar or curved surface. In some embodiments, the substrate comprises polydimethylsiloxane coated on a planar or curved surface.

In some embodiments, the substrate comprises polydimethylsiloxane coated on a glass substrate. In some embodiments, the substrate comprises polydimethylsiloxane coated on a Si substrate.

In some embodiments, the patterned layer comprises a polymeric material. In some embodiments, the patterned layer comprises an insulating material. In some embodiments, the patterned layer comprises an elastic material. In some embodiments, the patterned layer comprises polydimethylsiloxane.

In some embodiments, the patterned layer has an average or mean thickness of about 1-500 μm, or about 10-200 μm, or about 10-20 μm, or about 20-50 μm, or about 50-100 μm, or about 100-200 μm.

In some embodiments, the pillars comprise a biocompatible material. In some embodiments, the patterned layer comprises an elastic material. In some embodiments, the pillars comprise SU-8. In some embodiments, the pillars comprise polyimide.

In some embodiments, the pillars are adapted to transmit/magnify the force generated by the cells cultured on the top of pillars to the MSGs) disposed underneath the patterned layer.

In some embodiments, the pillars have an average or mean length of about 1-20 μm, or about 1-5 μm, or about 5-10 μm, or about 10-20 μm. In some embodiments, the pillars have an average or mean diameter of about 2-10 μm, or about 2-5 μm, or about 5-10 μm. In some embodiments, the pillars have an average or mean pitch of about 5-200 μm, or about 5-20 μm, or about 20-50 μm, or about 50-100 μm, or about 100-200 μm.

In some embodiments, the pillars and the patterned layer are physically or chemically or covalently bonded together.

In some embodiments, one or more or all of the microelectrodes are not covered by the pillars.

In some embodiments, the cardiac platform further comprises one or more eukaryotic cells and/or prokaryotic cells disposed on the pillars, wherein the contraction force of the cells are detectable by the micro-strain gauges, and wherein electrophysiology the cells are detectable by the microelectrodes. In some embodiments, the cardiac platform further comprises one or more mammalian cells disposed on the pillars. In some embodiments, the cardiac platform further comprises one or more murine cells disposed on the pillars. In some embodiments, the cardiac platform further comprises one or more human cells disposed on the pillars. In some embodiments, the cardiac platform further comprises one or more stem cells and/or progenitor cells disposed on the pillars.

In some embodiments, the cardiac platform further comprises one or more cardiomyocytes, cardiac stem cells and/or cardiac progenitor cells disposed on the pillars, wherein the contraction force of the cardiomyocytes, cardiac stem cells and/or cardiac progenitor cells are detectable by the micro-strain gauges, and wherein electrophysiology the cardiomyocytes, cardiac stem cells and/or cardiac progenitor cells are detectable by the microelectrodes. In some embodiments, the cardiac platform further comprises one or more murine cardiomyocytes, cardiac stem cells and/or cardiac progenitor cells disposed on the pillars. In some embodiments, the cardiac platform further comprises one or more human cardiomyocytes, cardiac stem cells and/or cardiac progenitor cells disposed on the pillars.

In some embodiments, in addition to cardiomyocytes, the cardiac platform further comprises one or more supporting fibroblast disposed on the pillars.

In some embodiments, the cardiac platform further comprises a beating cardiac tissue disposed on the pillars, wherein the contraction force of the beating cardiac tissue are detectable by the micro-strain gauges, and wherein electrophysiology the beating cardiac tissue are detectable by the microelectrodes. In some embodiments, the cardiac platform further comprises a beating murine cardiac tissue disposed on the pillars. In some embodiments, the cardiac platform further comprises a beating human cardiac tissue disposed on the pillars

Another aspect of some embodiments of the invention described herein relates to a method for culturing a cardiac tissue, comprising seeding one or more cardiac cells onto the cardiac platform described herein.

In some embodiments, the cardiac cells are seeded on the pillars. In some embodiments, the cardiac cells adhere to the pillars after being seeded.

In some embodiments, the method comprises seeding one or more cardiac stem cells and/or cardiac progenitor cells onto the cardiac platform. In some embodiments, the method comprises seeding one or more mammalian cardiac stem cells and/or cardiac progenitor cells onto the cardiac platform. In some embodiments, the method comprises seeding one or more murine cardiac stem cells and/or cardiac progenitor cells onto the cardiac platform. In some embodiments, the method comprises seeding one or more human cardiac stem cells and/or cardiac progenitor cells onto the cardiac platform.

In some embodiments, the method further comprises differentiating cardiac stem cells and/or cardiac progenitor cells disposed on the cardiac platform into cardiomyocytes. In some embodiments, the method further comprises differentiating mammalian cardiac stem cells and/or cardiac progenitor cells disposed on the cardiac platform into cardiomyocytes. In some embodiments, the method further comprises differentiating murine cardiac stem cells and/or cardiac progenitor cells disposed on the cardiac platform into cardiomyocytes. In some embodiments, the method further comprises differentiating human cardiac stem cells and/or cardiac progenitor cells disposed on the cardiac platform into cardiomyocytes.

In some embodiments, the method further comprises differentiating cardiac stem cells and/or cardiac progenitor cells disposed on the cardiac platform into a beating cardiac tissue. In some embodiments, the method further comprises differentiating mammalian cardiac stem cells and/or cardiac progenitor cells disposed on the cardiac platform into a beating cardiac tissue. In some embodiments, the method further comprises differentiating murine cardiac stem cells and/or cardiac progenitor cells disposed on the cardiac platform into a beating cardiac tissue. In some embodiments, the method further comprises differentiating human cardiac stem cells and/or cardiac progenitor cells disposed on the cardiac platform into a beating cardiac tissue.

In some embodiments, the method further comprises stimulating the cardiac cells disposed on the cardiac platform.

In some embodiments, the method further comprises exposing cardiac cells disposed on the cardiac platform to a drug compound. In some embodiments, the method further comprises exposing cardiac cells disposed on the cardiac platform to a biologic. In some embodiments, the method further comprises exposing cardiac cells disposed on the cardiac platform to a nucleic acid, a DNA, an RNA, an siRNA, an miRNA, a polypeptide, an antibody or fragment thereof, a cytokine, a growth factor, a toxin, a bacterial and/or a virus. In some embodiments, the method further comprises exposing the cardiac cells disposed on the cardiac platform to a transformation vector (e.g., a vector encoding a zinc finger protein, a transcription activator-like effector nucleases protein, or a CRISPR/Cas system).

In some embodiments, the method further comprises detecting contraction force of the cardiac cells by the micro-strain gauges. In some embodiments, the method further comprises detecting resistance changes of the micro-strain gauges.

In some embodiments, the method further comprises detecting electrophysiology of the cardiac cells by the microelectrodes.

A further aspect of some embodiments of the invention described herein relates to an method for detecting electrophysiology and contractility of cardiac cells or tissues, comprising: providing a cardiac platform comprising (a) a substrate layer which comprises a substrate and a plurality of micro-strain gauges and a plurality of microelectrodes disposed on the substrate, (b) a patterned layer disposed on the substrate layer which insulates the micro-strain gauges and exposes the microelectrodes, (c) a plurality of pillars disposed on the patterned layer, and (d) cardiac cells or tissues disposed on the pillars; and detecting the electrophysiology of the cardiac cells or tissues using the microelectrodes and detecting the contractility of the cardiac cells or tissues using the micro-strain gauges.

An additional aspect of some embodiments of the invention described herein relates to a method for fabricating the cardiac platform described herein, comprising patterning a plurality of micro-strain gauges and a plurality of microelectrodes onto a planar or curved substrate to obtain a subtrates layer, depositing a patterned layer onto the subtrates layer to insulate the micro-strain gauges and expose the microelectrodes, and depositing a plurality of pillars onto the patterned layer.

These and other features, together with the organization and manner of operation thereof, will become apparent from the following detailed description when taken in conjunction with the accompanying drawings.

### BRIEF DESCRIPTION OF THE DRAWINGS

FIG. 1: Schematic drawings of an example cardiac platform adapted for electrical recording of both electrophysiology and contraction of in vitro-cultured cardiomyocytes or cardiac tissues.

FIG. 2: Schematic drawings of individual components of an example cardiac platform.

FIG. 3: Photomask designs of example MSG geometries.

FIG. 4: MSGs on 4″ Si wafers pre-coated with PDMS.

FIG. 5: Electrical resistance of MSGs having different linewidths and side lengths.

FIG. 6: MSG geometries having different size and reproducibility.

FIG. 7: MSG geometries having different size and reproducibility.

FIG. 8: SEM images comparing the surfaces of different MSGs on PDMS.

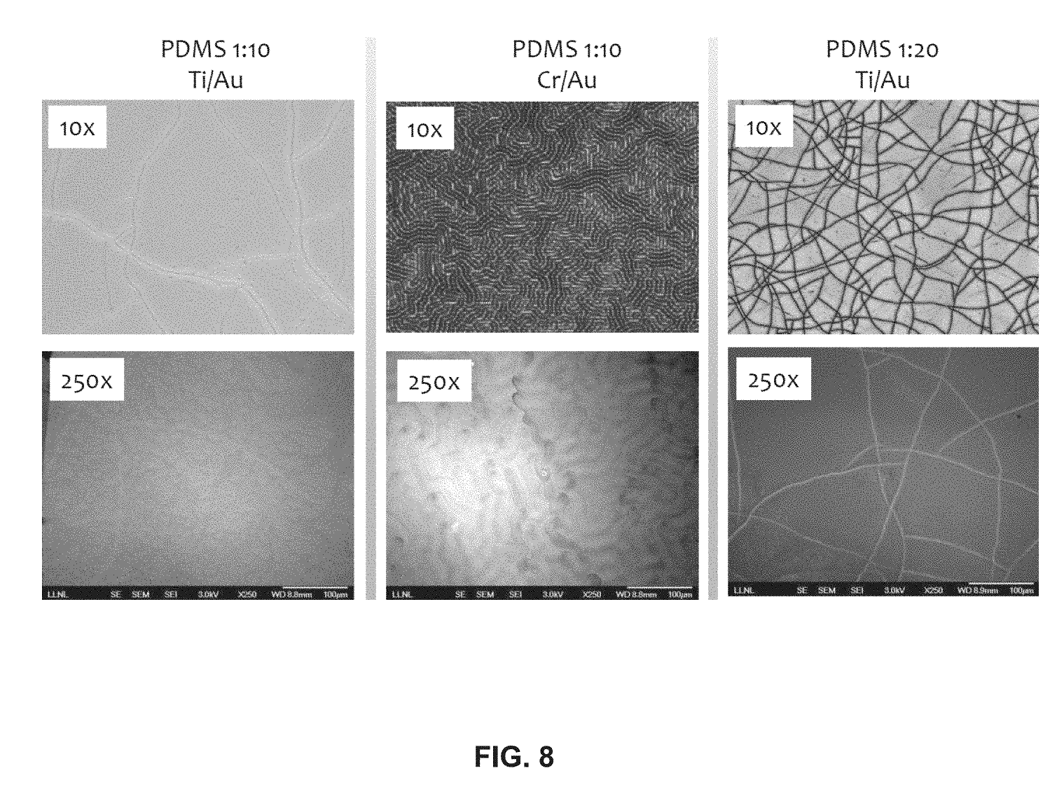

FIG. 9: SEM images comparing the surfaces of different MSGs on PDMS.

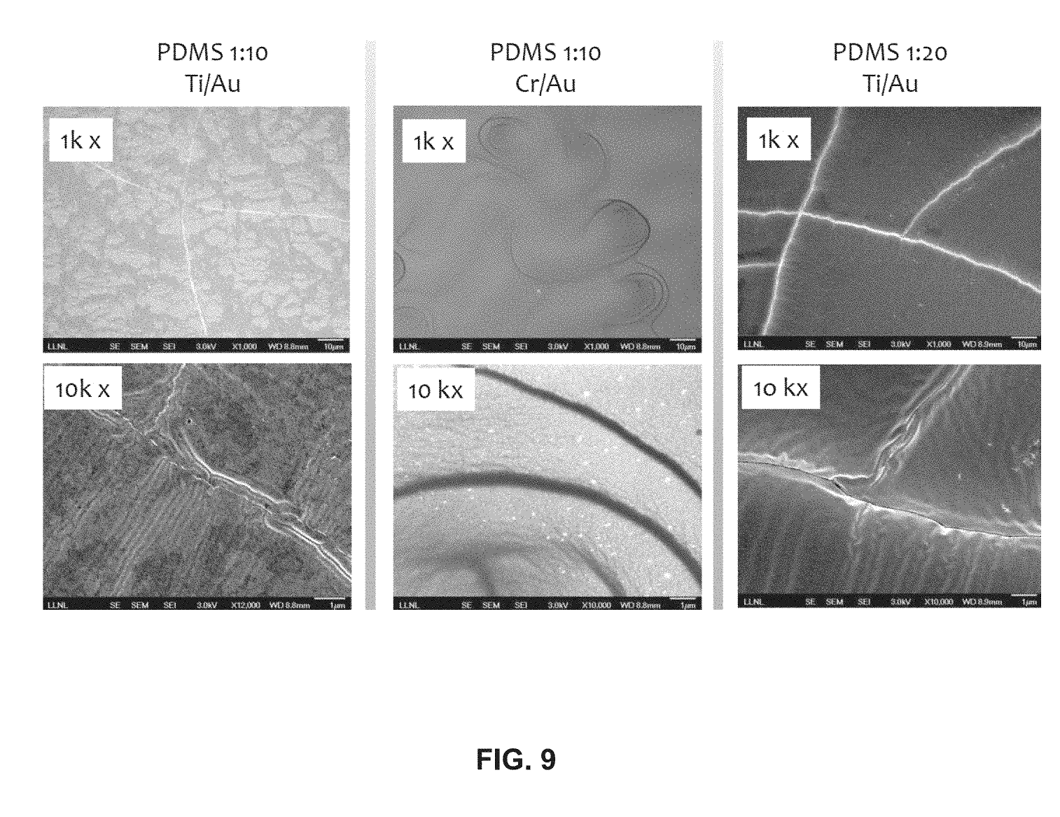

### DETAILED DESCRIPTION

Reference will now be made in detail to some specific embodiments of the invention contemplated by the inventors for carrying out the invention. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

In the following description, numerous specific details are set forth in order to provide a thorough understanding of the present invention. Particular example embodiments of the present invention may be implemented without some or all of these specific details. In other instances, well known process operations have not been described in detail in order not to unnecessarily obscure the present invention.

Various techniques and mechanisms of the present invention will sometimes be described in singular form for clarity. However, it should be noted that some embodiments include multiple iterations of a technique or multiple instantiations of a mechanism unless noted otherwise.

Healthy cardiac cells exhibit action potential generation and contraction phenotype at the single cell level. In contrast, damaged or dysfunctional cells often show altered patterns in electrophysiology and beating behavior. In order to obtain information on the electrical and mechanical properties of cardiac cells, disclosed herein is a novel cardiac platform allowing electrical recording of both electrophysiology and contractility of cardiac cells/tissues in 2D and 3D in vitro culture.

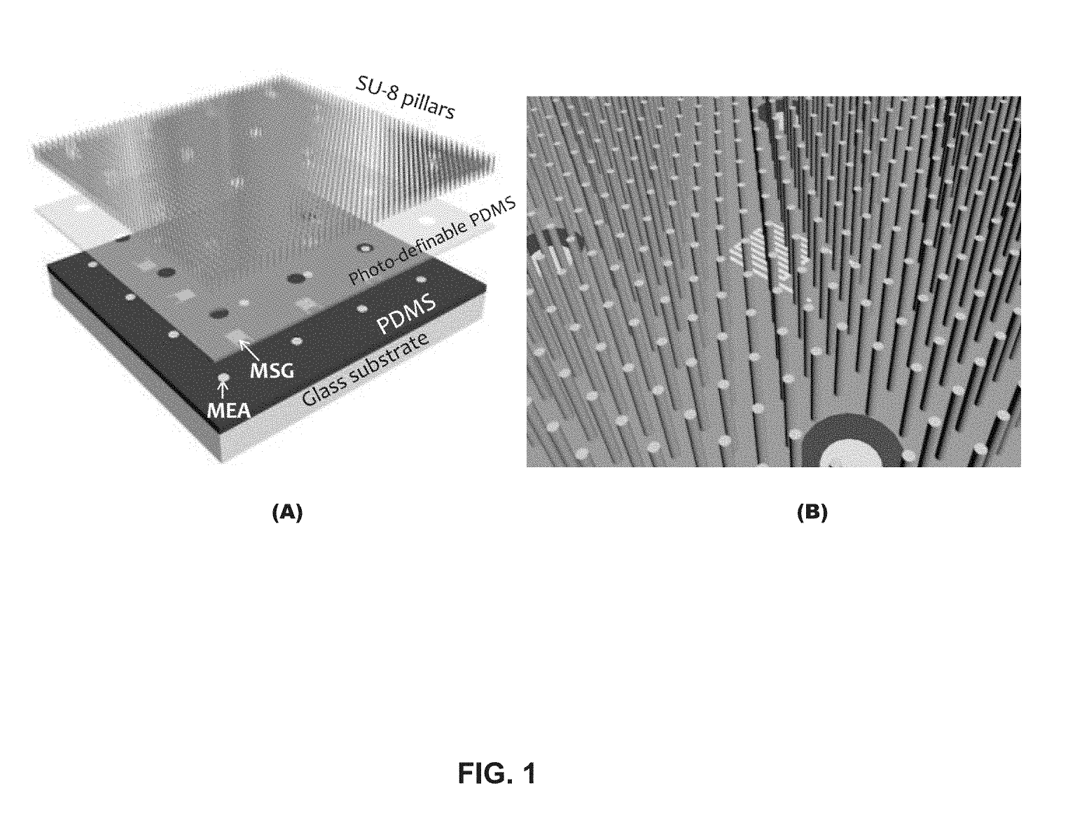

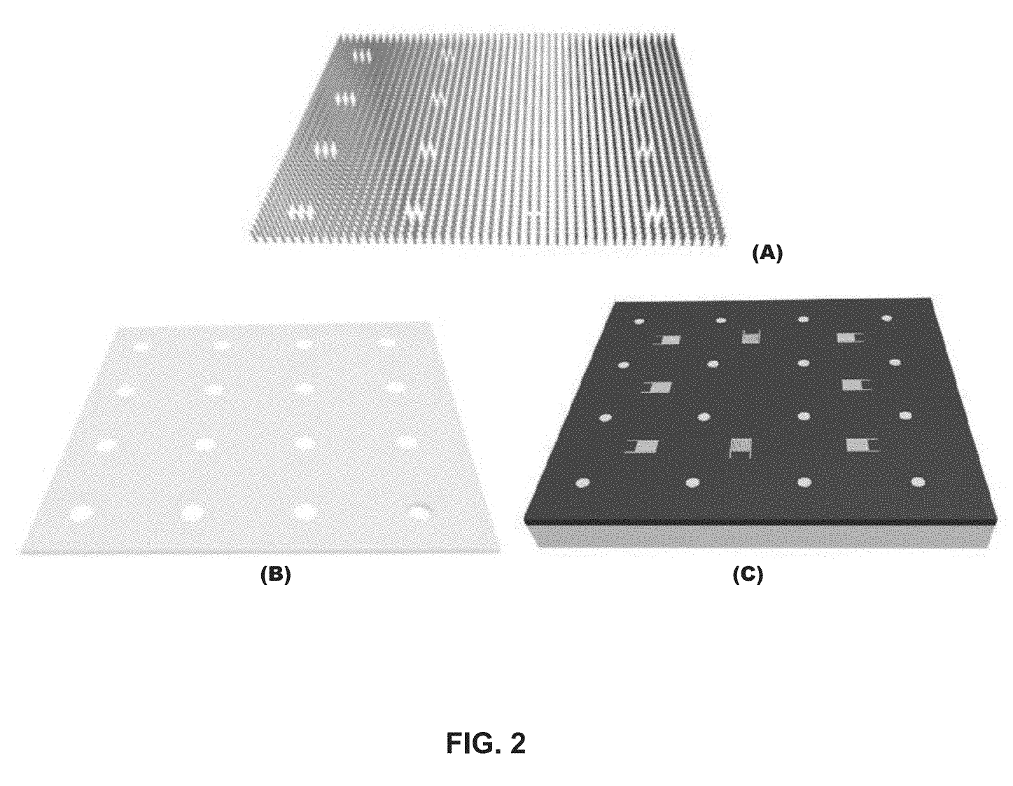

Cardiac Platform for Electrical Recording of Electrophysiology and Contractility of Cardiac Tissues. As shown in FIGS. 1 and 2, in some embodiments, the cardiac platform described herein comprises at least the following three components from the bottom to the top: (A) a planar substrate (e.g., a substrate obtainable or obtained by coating soft PDMS on a glass wafer) with micro-fabricated microelectrode arrays (MEAs) and micro-strain gauges (MSGs); (B) a patterned PDMS layer which exposes the MEAs but covers the MSGs; and (C) a SU-8 pillar arrays fabricated on the patterned PDMS layer. Cardiac cell can be seeded and grown on top of the SU-8 pillar arrays.

In the cardiac platform described herein, the SU-8 pillars can serve to transmit or magnify the force generated by cells (on the top of pillars) to the MSGs (at the bottom of pillars). Moreover, the MSGs can be used to detect the local deformation of the PDMS caused by the SU-8 pillar/cell contraction. Furthermore, the MEAs can be used to detect the extracellular electric potential correlated with action potential generation.

The cardiac platform described herein can be employed to simultaneously detect the electrophysiology and contraction properties of various cardiac systems, including neonatal/adult rat ventricular cardiomyocytes, human/rat induced pluripotent stem cells derived cardiomyocytes, and primary human cardiac tissues. Using this cardiac platform, comprehensive information can be obtained concerning the physiology, function and tissue damage development of cardiac systems, as well as cellular responses to drug stimuli.

In some embodiments, the cardiac platform can simultaneously and electrically record the action potential and contraction force of cardiac cells/tissues. For example, the MEAs can be exposed to the medium to directly interfere with the membrane potential. The stiff SU-8 pillar array can serve as cantilever to transmit/magnify the force generated by the cells to the underlying MSGs. The MSGs can be covered by a thin layer of photo-definable PDMS to insulate them from the medium while still allowing them to deform if the PDMS deforms.

In some embodiments, the patterned PDMS layer is obtainable or obtained by photolithography using photo-definable PDMS.

In some embodiments, both the substrate layer and the patterned layer comprise PDMS. For example, the substrate layer can comprise a PDMS-coated substrate, while the patterned layer can be composed of PDMS. In some embodiments, the two PDMS layers are physically or chemically or covalently bonded together. In some embodiments, the pillars are physically or chemically or covalently bonded to the patterned PDMS layer.

Concerning the detection of cellular contraction force of the cardiac cells/tissues cultured on the pillars, the cardiac platform described herein allows the cellular contraction force to be converted to a mechanical deformation or strain which is transmitted from the pillars to the MSGs, wherein the strain of the MSGs can be converted to electrical signals such as electrical resistance. Accordingly, to achieve electrical recording of cellular contraction force, the cardiac platform can employ a soft substrate that can deform under a small mechanical force, metal stain gauges fabricated on the soft substrate to detect the strain/deformation, and stiff micro-pillars serving as cantilevers to transmit and magnify the force.

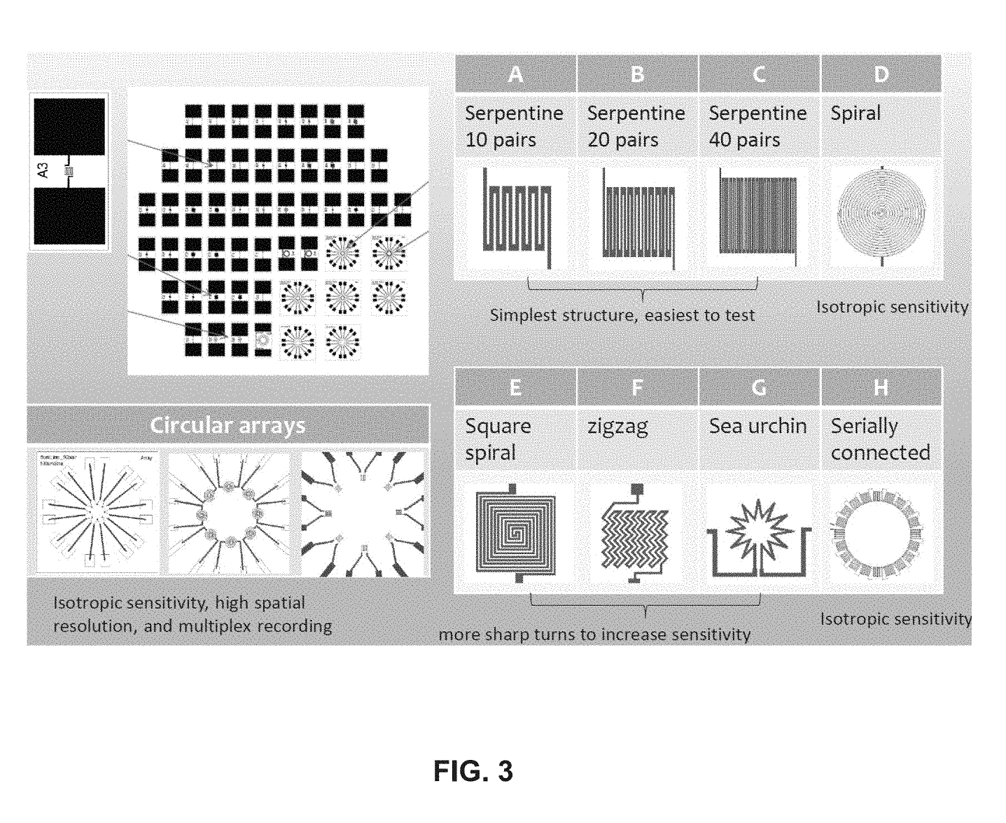

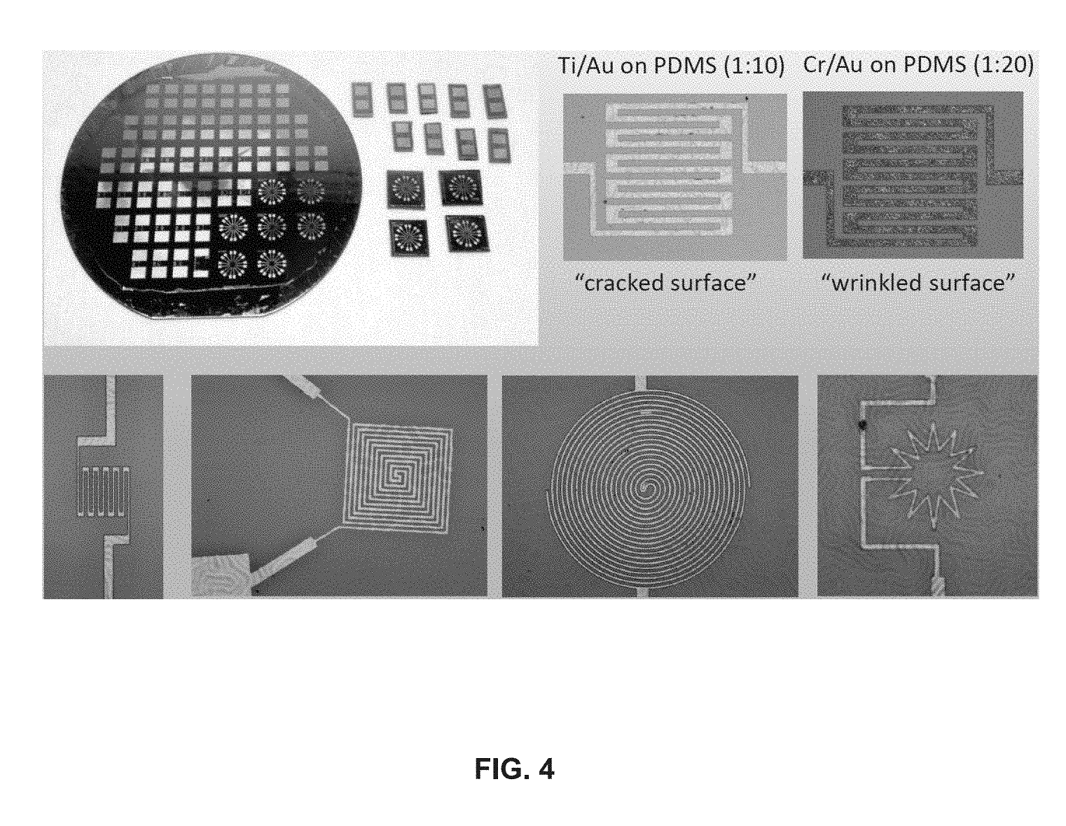

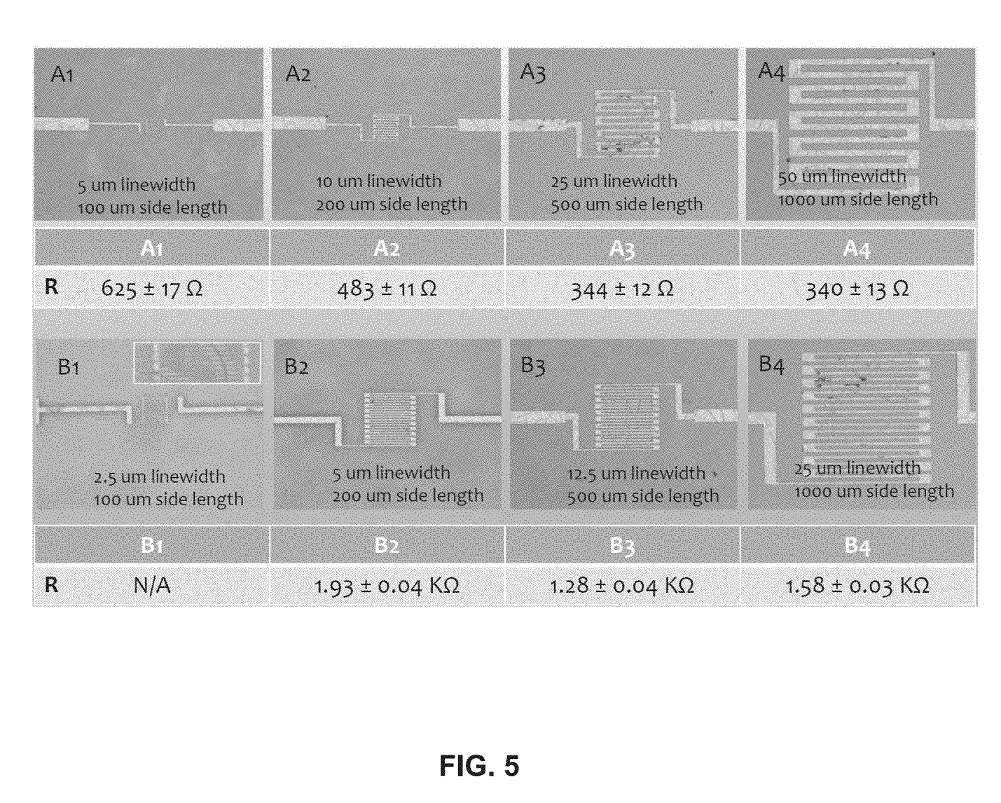

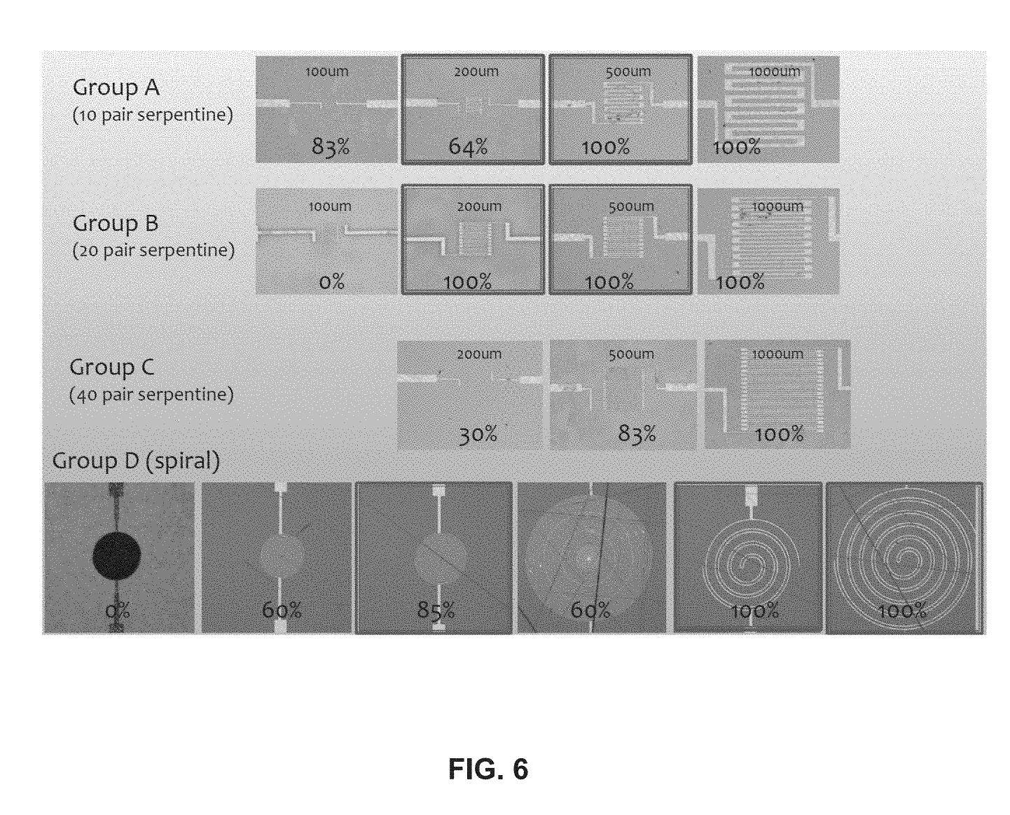

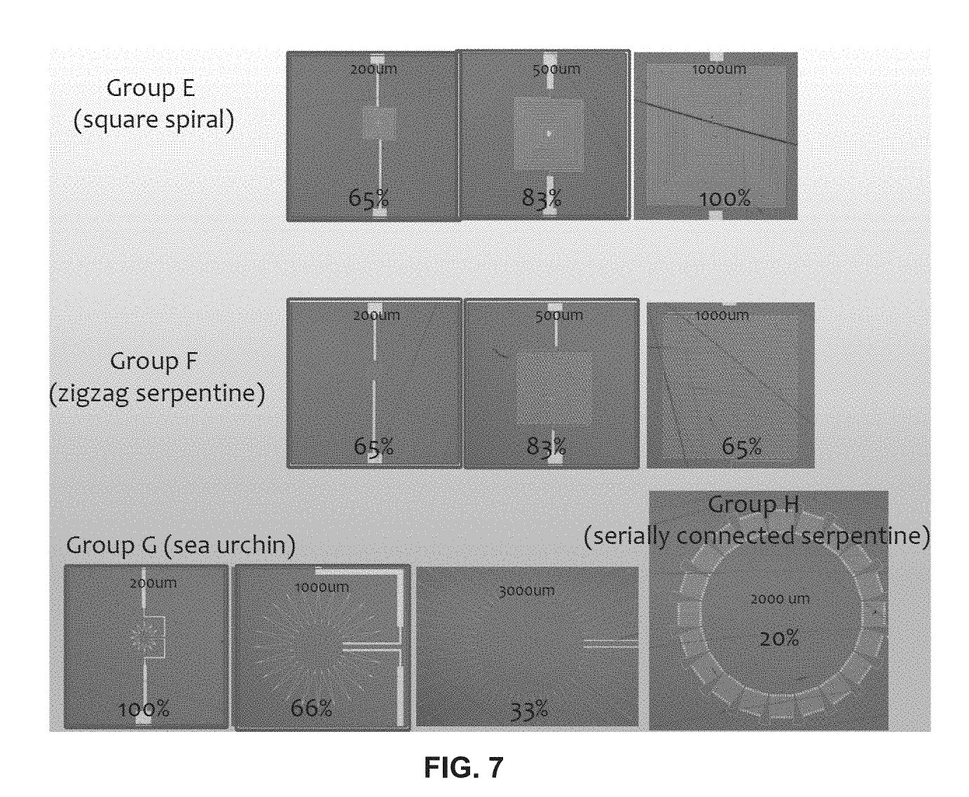

The MSGs can have various geometries, linewidths and side lengths, as shown in FIGS. 3-7. The MSGs can also have various electrical resistance in accordance with the requirements of specific applications. The resistance, R, can be determined by the device shape and the metal resistivity (ρ), according to R=ρ(L/A), where A and L are the cross-sectional area and effective length, respectively, of the electrical path. Once a tensile (or compressive) strain, ε, is applied along the longitudinal axis, L is increased (or decreased) due to the shape change, yielding a linearly increased (or decreased) resistance change, ΔR. The sensitivity of a strain gauge can be assessed by gauge factor (GF), where GF=(ΔR/R)/ε.

In some embodiments, the MSGs have isotropic sensitivity, high spatial resolution, and/or multiplex recording capability.

Applications of Cardiac Platform. The cardiac platform described herein can be used in a variety of applications. For example, they can be used in in-vitro cell/tissue culture, drug screening, pharmaceutical testing, tissue surrogates, drug delivery, toxicology test, pharmacology test, electrical stimulation and recording, optical imaging, cardiac beating assay, and human-relevant tissue models for drug testing.

### WORKING EXAMPLES

Fabrication Process of Cardiac Pillar Platform. The cardiac platform described herein, which are capable of electrical recording of both electrophysiology and contractility of cardiac cells/tissues, can be fabricated according to the following process:

1. Clean glass wafer with Piranha solution, followed deionized (DI) water rinse and nitrogen gas dry.

2. Spin coat PDMS (Sylgard 184, 1:10) at 500 rpm over the wafer. Wait until the surface flattens. Remove edge beads using a razor blade. Then bake the wafer on a hot plate set at 150° C.

3. Metal deposition using E-Beam deposition tool of Ti/Au=20/100 nm.

4. Spin coat photoresist onto the wafer, followed by photolithography through the 1st photomask that carrying electrode patterns. Develop the photoresist until field clears. Rinse and dry.

5. Immerse wafer in gold etch solution until field turns brown. Immerse wafer in 1:100 HF dip solution until field clears. Rinse and dry.

6. Remove photoresist using PRS2000 stripper.

7. Spin coat PDMS at 1000 rpm over the wafer. Wait until surface flattens. Remove edge beads using a razor blade. Then bake the wafer on a hot plate set at 150° C.

8. Evaporate a Ni thin film (100 nm) onto PDMS, followed by spin coat photoresist soft back, and photolithography through the 2nd photomask that expose the microdisks (for ephys microelectrode) and the external leads. Develop the photoresist, etch exposed Ni layer using Ni etchant. Then remove photoresist as in step 6. A nickel mask is formed on the top PDMS layer.

9. Dry etch PDMS layer to selectively expose ephys microelectrode and contact lead, as defined by the Ni mask. Then remove Ni layer using Ni etchant.

10. Oxygen plasma clean the wafer at 300 W for 3 min. This turns the surface of PDMS from hydrophobic to hydrophilic.

11. Spin coat SU-8 2010. Soft bake at 65° C. for 2 min, followed by photolithography using a 3rd photomask that carries micropillar pattern everywhere except the ephys electrode region. Then post bake, develop using SU-8 developer to generate the micro-pillar pattern. Hard bake at 200° C. is optional.

12. Gently rinse the wafer with ethanol and dry.

As used herein, the singular terms “a,” “an,” and “the” include plural referents unless the context clearly dictates otherwise. Thus, for example, reference to a compound can include multiple compounds unless the context clearly dictates otherwise.

As used herein, the terms “substantially,” “substantial,” and “about” are used to describe and account for small variations. When used in conjunction with an event or circumstance, the terms can refer to instances in which the event or circumstance occurs precisely as well as instances in which the event or circumstance occurs to a close approximation. For example, the terms can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations, which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scopes of this invention.

What is claimed is:

1. A cardiac platform, comprising a substrate layer comprising a substrate and a plurality of micro-strain gauges and a plurality of microelectrodes disposed on the substrate, a patterned layer disposed on the substrate layer which insulates the micro-strain gauges and exposes the microelectrodes, and a plurality of pillars disposed on the patterned layer.

2. The cardiac platform of claim 1, wherein the microelectrodes are adapted to detect cardiac electrophysiology.

3. The cardiac platform of claim 1, wherein the micro-strain gauges are adapted to detect contraction force transmitted through the pillars and the patterned layer.

4. The cardiac platform of claim 1, wherein the micro-strain gauges comprise at least one metal or metal compound.

5. The cardiac platform of claim 1, wherein the substrate comprises a polymeric material coated on a planar surface.

6. The cardiac platform of claim 1, wherein the substrate comprises polydimethylsiloxane coated on a glass wafer.

7. The cardiac platform of claim 1, wherein the patterned layer comprises a polymeric material.

8. The cardiac platform of claim 1, wherein the patterned layer comprises polydimethylsiloxane.

9. The cardiac platform of claim 1, wherein the pillars comprise a biocompatible material.

10. The cardiac platform of claim 1, wherein the pillars comprise SU-8.

11. The cardiac platform of claim 1, further comprising one or more cardiomyocytes, cardiac stem cells and/or cardiac progenitor cells disposed on the pillars, wherein the contraction force of the cardiomyocytes, cardiac stem cells and/or cardiac progenitor cells are detectable by the micro-strain gauges, and wherein electrophysiology the cardiomyocytes, cardiac stem cells and/or cardiac progenitor cells are detectable by the microelectrodes.

12. The cardiac platform of claim 1, further comprising a beating cardiac tissue disposed on the pillars, wherein the contraction force of the beating cardiac tissue are detectable by the micro-strain gauges and wherein electrophysiology the beating cardiac tissue are detectable by the microelectrodes.

13. A method for culturing a cardiac tissue, comprising seeding one or more cardiac cells on the cardiac platform of claim 1.

14. The method of claim 13, wherein the cardiac cells adhere onto the pillars.

15. The method of claim 13, wherein the cardiac cells comprise cardiac stem cells and/or cardiac progenitor cells.

16. The method of claim 15, further comprising differentiating the cardiac stem cells and/or cardiac progenitor cells into cardiomyocytes.

17. The method of claim 15, further comprising differentiating the cardiac stem cells and/or cardiac progenitor cells into a beating cardiac tissue.

18. The method of claim 13, further comprising stimulating the cardiac cells with a drug compound.

19. The method of claim 13, further comprising detecting contraction force of the cardiac cells by the micro-strain gauges and detecting electrophysiology of the cardiac cells by the microelectrodes.

20. A method for detecting electrophysiology and contractility of cardiac cells or tissues, comprising:
providing a cardiac platform comprising (a) a substrate layer which comprises a substrate and a plurality of micro-strain gauges and a plurality of microelectrodes disposed on the substrate, (b) a patterned layer disposed on the substrate layer which insulates the micro-strain gauges and exposes the microelectrodes, (c) a plurality of pillars disposed on the patterned layer, and (d) cardiac cells or tissues disposed on the pillars; and
detecting the electrophysiology of the cardiac cells or tissues using the microelectrodes and detecting the contractility of the cardiac cells or tissues using the micro-strain gauges.
